(19) United States
(12) Patent Application Publication
(10) Pub. No.: US 2007/0213749 A1
(43) Pub. Date: Sep. 13, 2007

(54) Medical procedure performed inside abdominal cavity

(75) Inventors: Takahiro Kogasaka, Tokyo (JP); Takayasu Mikkaichi, Tokyo (JP); Kensei Nakahashi, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 11/371,456

(22) Filed: Mar. 8, 2006

(51) Int. Cl. A61B 17/08

(52) U.S. Cl. 606/153

(57) ABSTRACT

A medical procedure performed inside an abdominal cavity includes: performing a first treatment at a target position inside the abdominal cavity by a first apparatus that has been introduced percutaneously into the abdominal cavity; and performing a second treatment, using a second apparatus that has been introduced into the abdominal cavity via a natural aperture of a living body, in cooperation with the first apparatus inside the abdominal cavity, or alternatively, performing a second treatment that is necessitated as a result of the first treatment being performed after the first treatment has been performed.

### BACKGROUND OF THE INVENTION

1. Field of the Invention

The present invention relates to a medical procedure to perform a desired treatment inside an abdominal cavity.

2. Description of Related Art

Treatment such as cutting, extirpating, and suturing a predetermined position of an organ inside an abdominal cavity is achieved by performing an abdominal operation or by performing a laparoscopic operation in which a plurality of insertion holes are formed in an abdominal wall and treatment instruments such as a laparoscope and forceps are then inserted through these holes. A laparoscopic operation (also known as a laparoscopic surgery operation) is a medical procedure in which a plurality of narrow tubes known as trocars are passed into an abdomen, and then a laparoscope or treatment instruments are inserted into the abdominal cavity via the trocars. Specifically, for example, the removal of a gall bladder, an appendectomy, a gastrectomy, total gastric resection, or subtotal gastrectomy to counter the early stages of gastric cancer, a colon excision or small bowel excision to counter colonic or rectal cancer, or splenectomy can be achieved by performing a laparoscopic surgery operation. If the organ or lesioned portion to be extirpated (i.e., removed to the body exterior) is large in size and cannot be retrieved via a trocar, then an abdominal operation to make an incision in the abdomen is performed in order to allow retrieval.

Compared with an abdominal operation, the size of the abdominal incision is smaller in a laparoscopic surgery operation. Therefore, laparoscopic surgery has advantages over an abdominal operation in that less pain is felt by the patient after the operation, recovery after the operation is quicker (i.e., the patient is able to return to work sooner), and there is less scarring resulting in less disfiguration. FIG. 1 of United States Pre-grant Patent Publication No. 2005/0222534 shows an example of this type of laparoscopic surgery operation.

### SUMMARY OF THE INVENTION

It is an object of the present invention to provide a medical procedure to be performed in an abdominal cavity that, when treating a predetermined location in an abdominal cavity, restricts to a minimum the size of an insertion hole that is formed in an abdominal wall in order to allow treatment instruments and the like to be inserted, and that lessens the burden on a patient, and that causes the minimum disfiguration after an operation.

The medical procedure performed inside an abdominal cavity of the present invention includes: performing a first treatment at a target position inside the abdominal cavity by a first apparatus that has been introduced percutaneously into the abdominal cavity; and performing a second treatment, using a second apparatus that has been introduced into the abdominal cavity via a natural aperture of a living body, in cooperation with the first apparatus inside the abdominal cavity, or alternatively, performing a second treatment that is necessitated as a result of the first treatment being performed after the first treatment has been performed.

### BRIEF DESCRIPTION OF THE DRAWINGS

FIG. 1 is an explanatory view illustrating the cutting of a lesioned portion according to an embodiment.

FIG. 2 is an explanatory view illustrating the cutting of a lesioned portion according to the embodiment.

FIG. 3 is an explanatory view illustrating the retrieval and removal to the body exterior of a lesioned portion according to the embodiment.

FIG. 4 is an explanatory view illustrating the retrieval and removal to the body exterior of a lesioned portion according to the embodiment.

FIG. 5 is an explanatory view illustrating the retrieval and removal to the body exterior of a lesioned portion according to the embodiment.

FIG. 6 is an explanatory view illustrating the suturing of a through hole according to the embodiment.

FIG. 7 is an explanatory view illustrating another example of the suturing of a through hole according to the embodiment.

FIG. 8 is an explanatory view illustrating the illustrating the cutting of a portion to be cut out according to a first variant example of the embodiment.

FIG. 9 is an enlarged perspective view of an automatic suturing and cutting instrument according to the first variant example of the embodiment. FIG. 10 is an explanatory view illustrating an anastomosis performed on a stomach and small bowel according to the first variant example of the embodiment.

FIG. 11 is an explanatory view illustrating an anastomosis performed on a stomach and small bowel according to the first variant example of the embodiment.

FIG. 12 is an enlarged perspective view of a suturing instrument according to the first variant example of the embodiment.

FIG. 13 is an explanatory view showing the detailed cutting out of an extirpated lesioned portion according to a second variant example of the embodiment.

FIG. 14 is an explanatory view showing the removal to the body exterior of a small section that has been cut from a lesioned portion according to the second variant example of the embodiment.

FIG. 15 is an overall view showing as an example of a suturing instrument a needle grasping instrument that is used when a predetermined position is sutured.

FIG. 16 is an enlarged view of the distal end portion shown in FIG. 15.

FIG. 17 is an overall view showing another example of a suturing instrument that is used when a predetermined position is sutured.

### DETAILED DESCRIPTION OF THE INVENTION

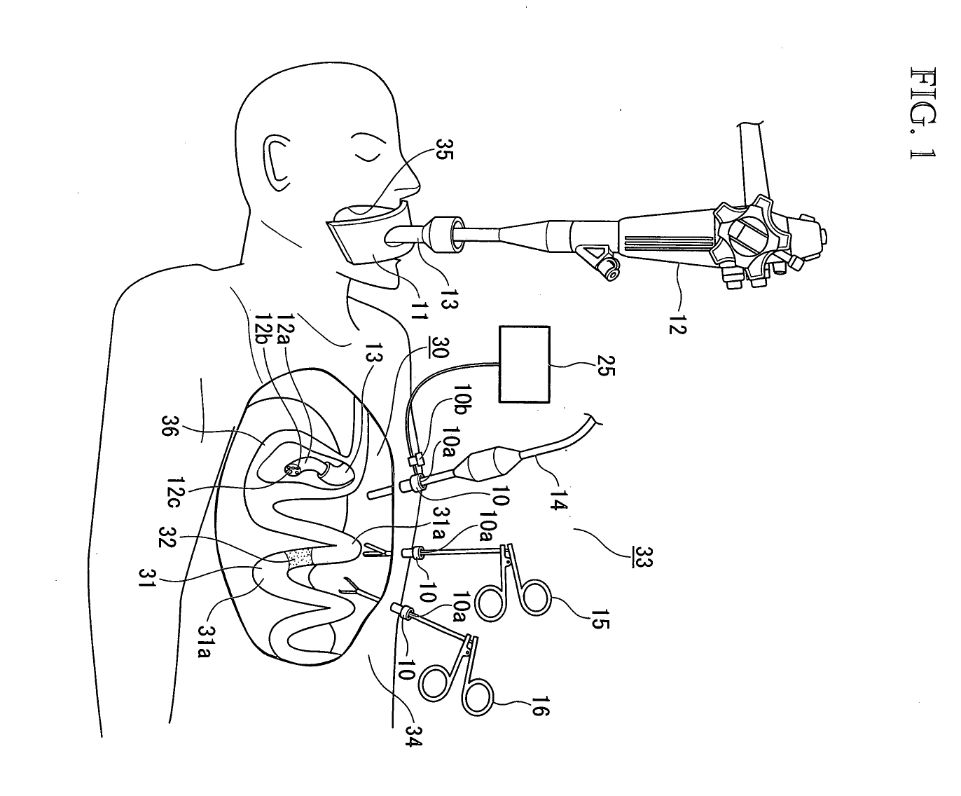

FIGS. 1 to 6 show a medical procedure performed inside an abdominal cavity according to the present embodiment In the present embodiment, a description is given of an example in which a lesioned portion 32 (i.e., an object for retrieval) that is a predetermined position (also referred to as a target position) of an organ, for example, a small bowel 31 or part thereof inside an abdominal cavity 30 is cut inside the abdominal cavity 30, and is then removed (i.e., extirpated) to a body exterior 33. However, the present embodiment is not limited to this and it may also be applied to the extirpation of a gall bladder or appendix, the extirpation of a lesioned portion of a liver, or the extirpation of another hollow organ such as a stomach, a colon, a duodenum or the like.

As shown in FIG. 1, firstly, trocars 10 are made to pierce predetermined locations of an abdominal wall 34 and are then left in place thereby forming insertion holes 10a that are used to insert grasping instruments such as forceps percutaneously inside the abdominal cavity 30. Note that the positions where the trocars 10 are inserted may be side abdomen portions or the like in the abdominal wall 34, and are appropriately set in accordance with the locations inside the abdominal cavity 30 of the predetermined positions where treatment is to be performed. In addition, the hole diameter of the insertion holes 10a for the trocars 10 to be used are set at 5 mm or less, and are at least big enough for the various forceps and laparoscopes to be inserted there through. Next, an insufflator 25 is connected to an air supply port 10b of a trocar 10, and the interior of the abdominal cavity 30 is inflated by being supplied with carbon dioxide gas or the like. Note that the insufflation may also be performed by inserting an insulation needle into the abdominal cavity 34. Note that the insufflation is performed in order to secure a space when performing the treatment illustrated below, and it is not essential for it to be performed provided that the desired space can be secured. Moreover, the method used to secure a space is not limited to the aforementioned insufflation and a method based on a known lifting method may also be used.

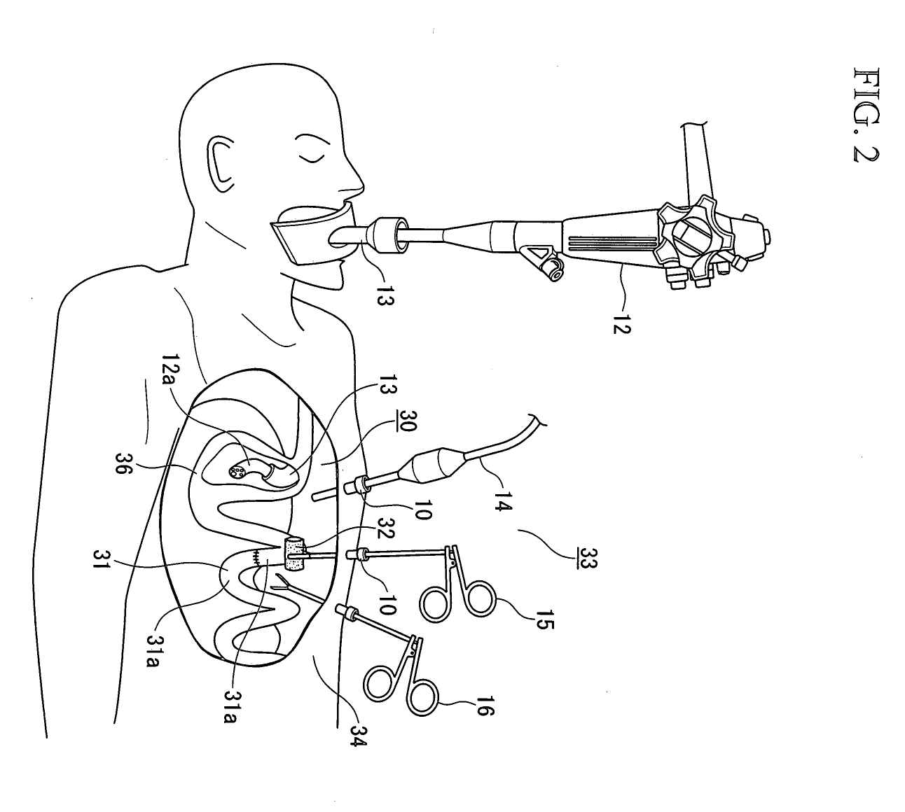

Firstly, in the first step of the treatment, a normal portion 31 a of the small bowel 31 is cut from the lesioned portion 32. More specifically, as shown in FIG. 1, a laparoscope 14 and first apparatuses in the form of grasping forceps 15 and shearing forceps 16 that serve as an incision instrument are inserted through the three stationary trocars 10 into the abdominal cavity 30. Here, the term “cut” refers to an action to create a state that allows a lesioned portion that has appeared on an organ or allows the organ itself to be extirpated to the outside of a living body (i.e., a state in which the relevant portion can be removed to the body exterior). Note that the incision instrument is not limited to the shearing forceps 16 and, depending on the objective, dissecting forceps or an electric scalpel can be selected as is appropriate. It is only necessary for the incision instrument to be able to be inserted percutaneously through a trocar 10 into the abdominal cavity 30 and then be able to cut a predetermined position. Next, as shown in FIG. 2. based on observations made using the laparoscope 14, the lesioned portion 32 is grasped by the grasping forceps 15 and the lesioned portion 32 is cut from the normal portion 31a of the small bowel 31 by the shearing forceps 16. Next, the shearing forceps 16 are replaced by a suturing instrument or grasping forceps or the like (not shown), and end portions of the remaining normal portion 31a of the small bowel 31 are sutured and connected together.

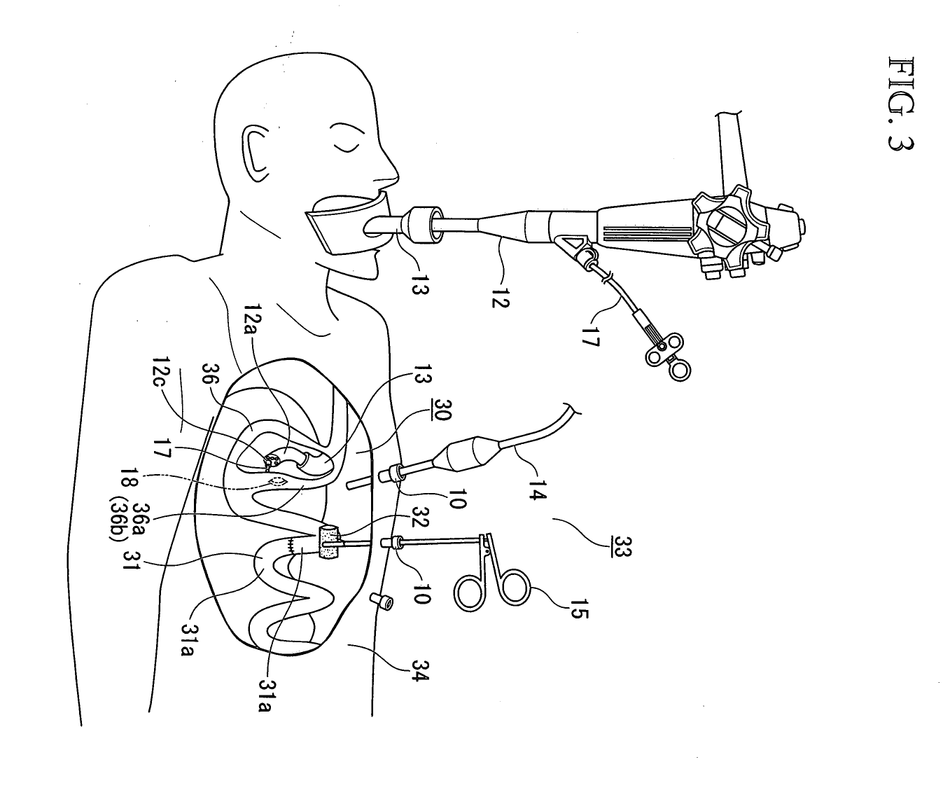

Next, in the second step of the treatment, the lesioned portion 32 of the small bowel 31 that was cut in the first step is removed to the body exterior 33. In the present embodiment, the removal of the lesioned portion 32 is performed through a natural aperture in the living body. More specifically, an aperture that communicates with the abdominal cavity is formed in a hollow organ (also referred to as a hollow internal organ) that communicates with a natural aperture of the living body. A retrieval instrument is introduced into the abdominal cavity through this formed aperture and the lesioned portion is retrieved. It is then moved to the inner side of the hollow organ and is extirpated through the natural aperture. The method used to form an aperture in these embodiments uses an endoscope 12 that has been inserted into the living body through the natural aperture of the living body. The endoscope 12 is inserted into the stomach 36 through a natural aperture in the form of the mouth 35 of a patient to which a mouthpiece 11 has been fitted, and a distal end 12a thereof is introduced into the abdominal cavity through a through hole 18 that is formed by making an incision in a stomach wall 36a. The through hole 18 is formed by inserting a high frequency knife 17 into a channel 12c in the endoscope 12 and observing the operation using an observation apparatus provided in the endoscope 12 (see FIG. 3). However, the method used to form the through hole 18 is not limited to the one described above and it is also possible to form the through hole 18 by making an incision from the outside of the stomach 36 (i.e., the abdominal cavity side of the hollow organ) using, for example, the shearing forceps 16 that have been inserted through a trocar 10. The through hole 18 is made large enough to enable the extirpated lesioned portion 32 to be retrieved. Moreover, the location where the through hole 18 is formed can be appropriately selected in accordance with the intended treatment, however, it is preferable for the location to be the front wall 36b of the stomach 36 (or an area on the forward side (i.e., the abdomen portion side) of the greater momentum that is hanging down lower than the greater curvature of the stomach 36) in consideration of the ease of approach of the endoscope 12 to the abdominal cavity 30.

Note that in the present embodiment an overtube 13 is also used when the endoscope 12 is being inserted into the living body. The overtube 13 is used as a guide tube to guide the insertion into and removal from the living body of a device having an insertion portion such as the endoscope 12, however, it is also possible to insert an apparatus into a living body without using the overtube 13. Moreover, when forming the through hole 18, air is supplied to the stomach interior from an air supply channel (namely, an air supply conduit that has been introduced into the body interior) 12b that is provided in the endoscope 12 and the stomach 36 is inflated.

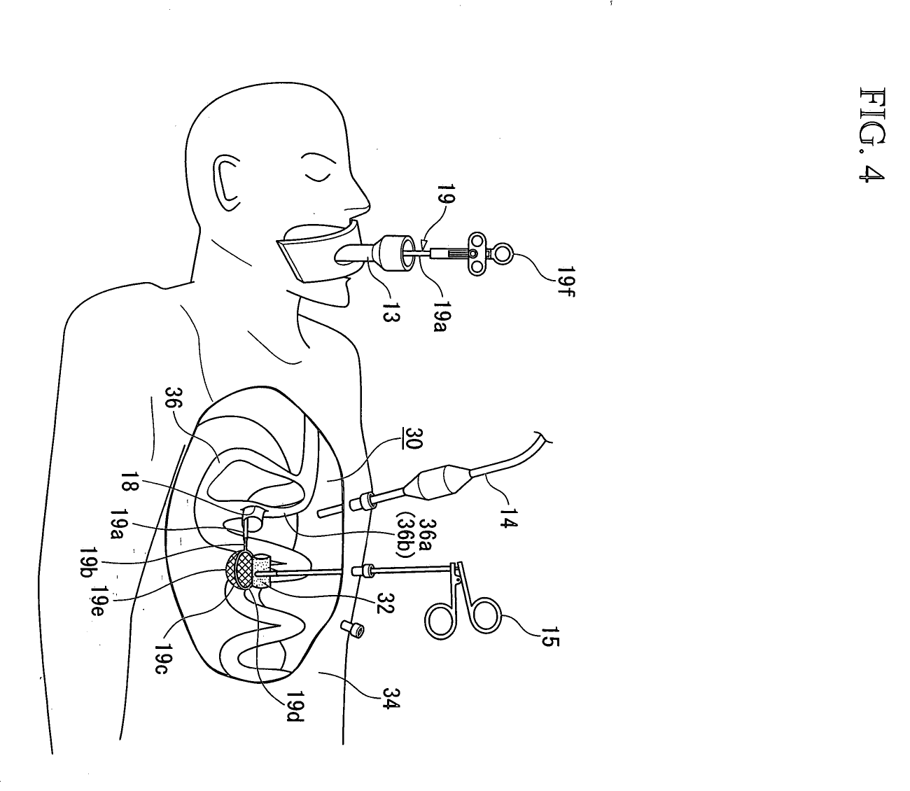

FIG. 4 shows a state in which a distal end of the overtube 13 has been introduced into the abdominal cavity 30 via the through hole 18, a retrieval instrument in the form of a retrieval net 19 has then been inserted through the interior of the overtube 13, and a retrieval portion 19c has been made to protrude from the distal end of the overtube 13.

The retrieval net 19 has a sheath 19a, an operating wire 19b that is inserted inside the sheath 19a, and the retrieval portion 19c that is provided at a distal end portion of the operating wire 19b and retrieves a desired object. The retrieval portion 19c has a toroidal wire 19d that has resiliency and is in a toroidal shape and is provided at a distal end portion of the operating wire 19b, and a net 19e that is suspended inside the toroidal wire 19d. An operating section 19f is provided at a proximal end portion of the sheath 19a and the operating wire 19b can be moved reciprocatingly inside the sheath 19a. The retrieval portion 19c is able to be accommodated together with the operating wire 19b inside the sheath 19a through an operation of the operating section 19f, and when the retrieval portion 19c is pushed out from the sheath 19a, it expands into a toroidal shape through its own resiliency. Note that the retrieval instrument is not limited to the retrieval net 19 and, instead of the net 19e, it is also possible to use a retrieval bag whose aperture is attached to the toroidal wire 19d.

The retrieval portion 19c of the retrieval net 19 is made to protrude from the distal end of the overtube 13 inside the abdominal cavity 30. The lesioned portion 32 that was cut in the first treatment step is then placed in the recovery portion 19c by the grasping forceps 15. In this state, by pulling the operating wire 19b to the proximal end side using the operating section 19f, the toroidal wire 19d of the retrieval portion 19c is also pulled into the sheath 19a. As a result, the extirpated lesioned portion 32 is enclosed in the net 19e of the retrieval portion 19c and is placed inside the retrieval portion 19c so that it cannot fall out.

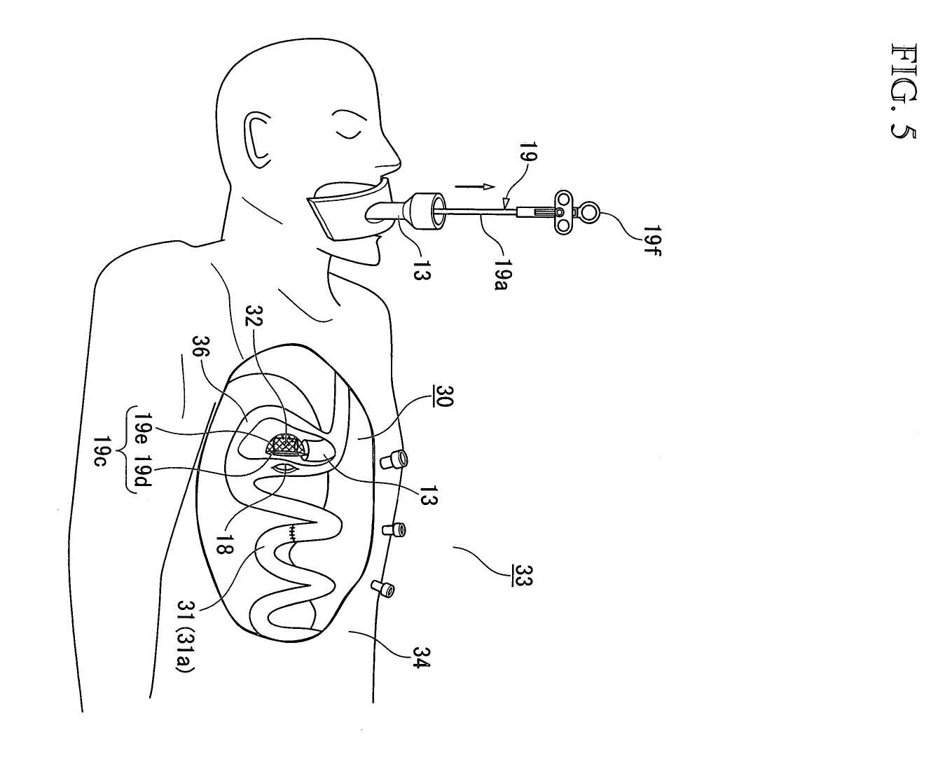

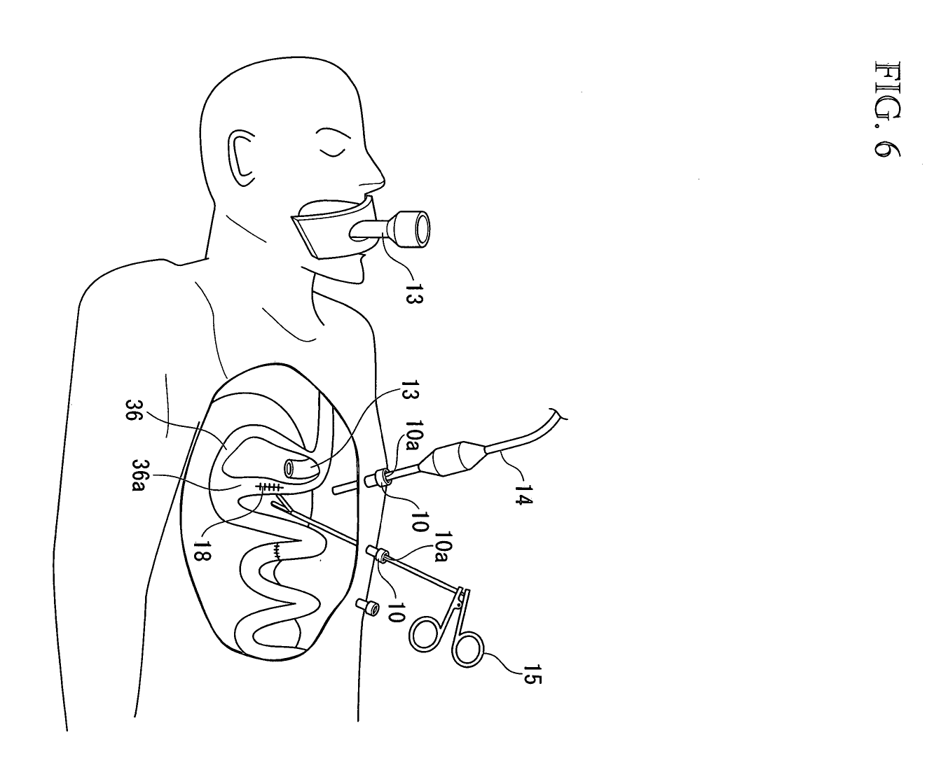

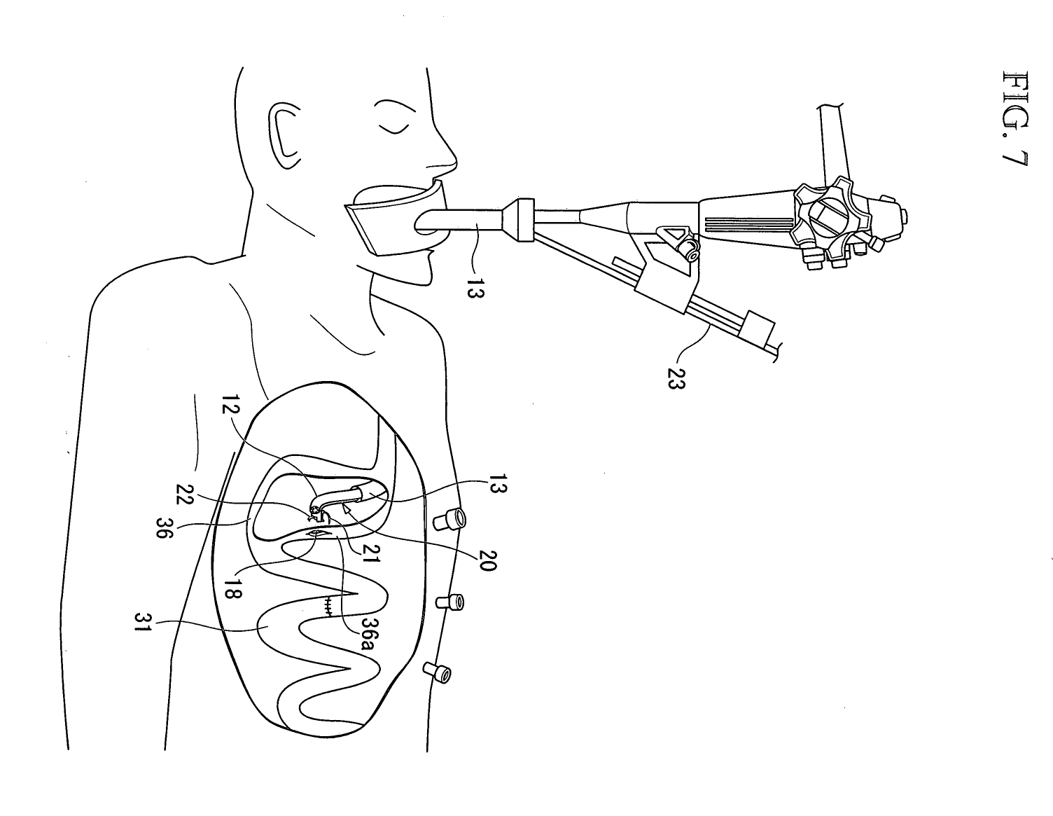

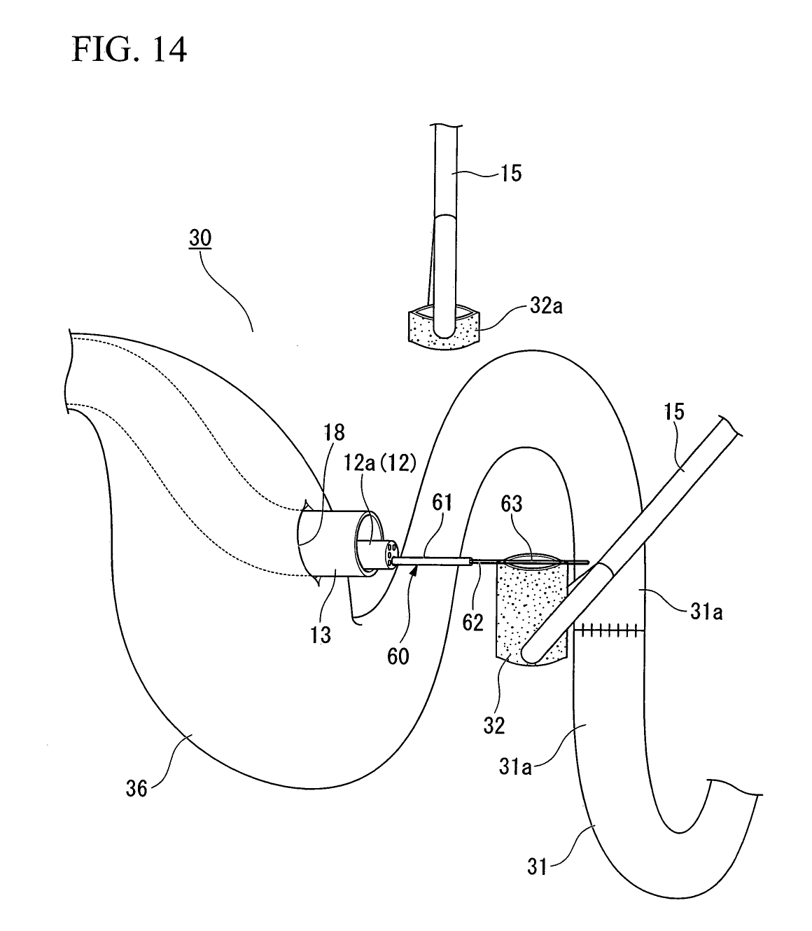

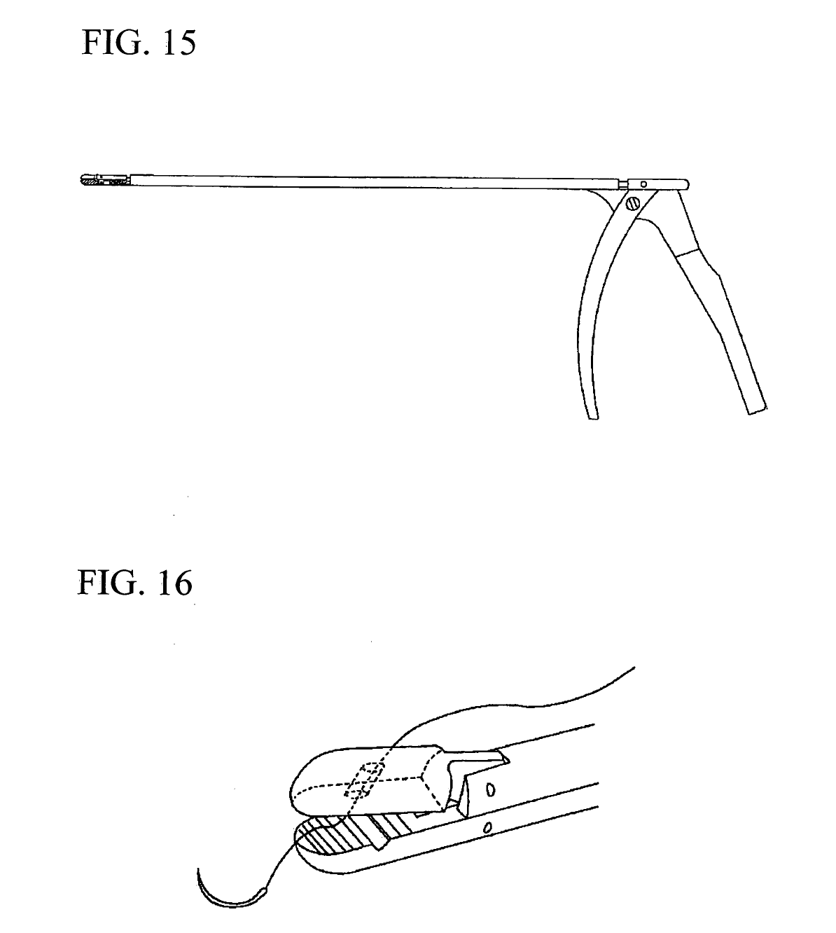

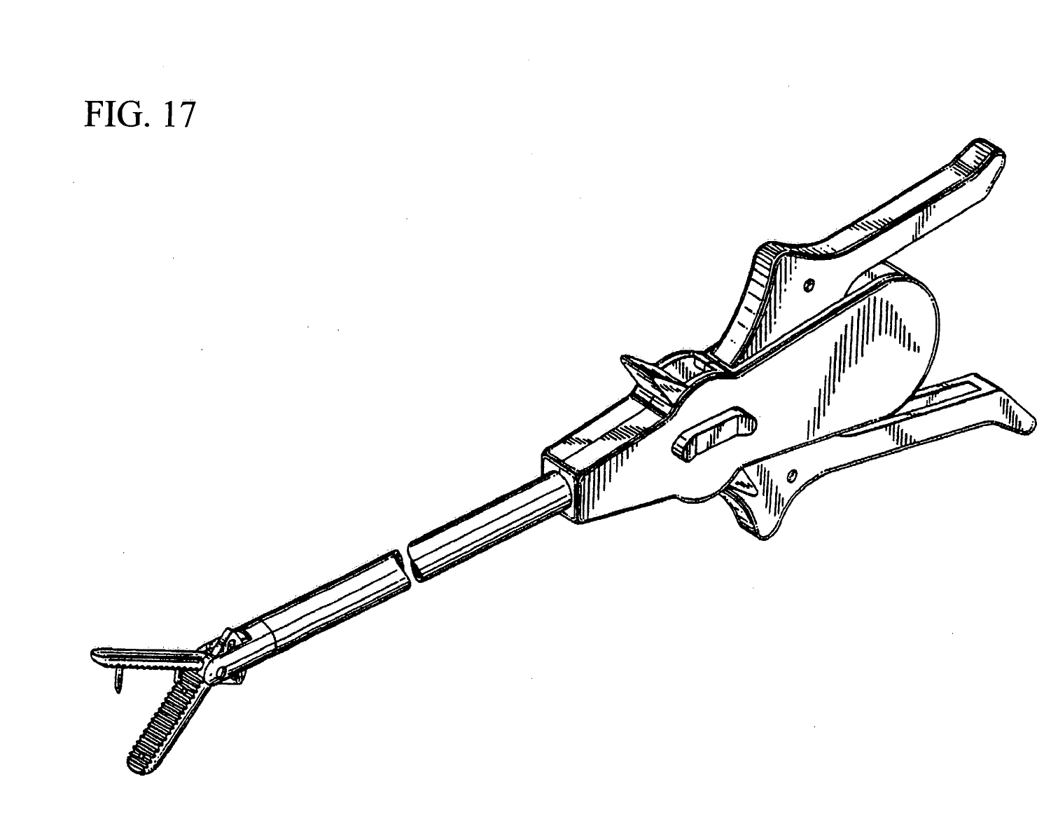

As shown in FIG. 5, by then drawing the retrieval net 19 to the proximal end side in this state, the lesioned portion 32 that has been retrieved to the retrieval portion 19c of the retrieval net 19 is removed to the body exterior 33 through the interior of the overtube 13. Lastly, needle forceps and suture thread having a needle attached thereto, such as are shown in FIGS. 15 and 16, or a suturing instrument such as that shown in FIG. 17 are introduced via the trocars 10 into the abdominal cavity 30. The through hole 18 that is formed in the stomach wall 36a of the stomach 36 is then sutured and closed up. An example of a suturing instrument is disclosed in Japanese Unexamined Patent Application, First Publication No. H09-84799 and U.S. Pat. No. 5,728,107, the entire contents of which are incorporated herein. FIG. 6 shows a state when the suturing has been completed. Note that the suturing of the through hole 18 may also be a method in which the suturing is performed from the interior portion (namely, the interior side of the hollow organ) of the stomach 36. FIG. 7 shows an example of this. Namely, a suturing apparatus 20 is placed alongside the outside of the endoscope 12 that has been inserted through the overtube 13. A needle and thread are attached to forceps components 21 and 22 that can be freely opened and closed at a distal end of the suturing apparatus 20. Namely, by opening and closing the forceps components 21 and 22 by operating an operating section 23 that is provided at a proximal end thereof, it is possible to suture the stomach wall 36a and close up the through hole 18. Note that when retrieving a retrieval object such as a lesioned portion or an organ, it is also possible to insert a retrieval instrument and an endoscope that has an observation apparatus inside the overtube 13, and then perform the retrieval task while verifying the action using the observation apparatus of the endoscope.

As described above, in the procedure of the present embodiment, it is not necessary to cut open the abdomen. Furthermore, it is possible to retrieve the cut lesioned portion 32 without forming an aperture in the abdominal wall 34 that matches the size of the object being recovered, but, instead, by passing it through a natural aperture via the through hole 18 that has been formed in the stomach 36. Because of this, it is possible to keep the diameter of the insertion hole 10a that is formed in the abdominal wall 34 to the minimum hole diameter size of 5 mm or less that enables at least the grasping forceps 15 and shearing forceps 16 to be inserted. Moreover, in the insertion hole 10a, because the hole diameter is small, it can be closed by natural closure without there being any need for suture closure. As a result, treatment can be performed that keeps the burden on a patient to the minimum, and scarring after the operation can be lessened.

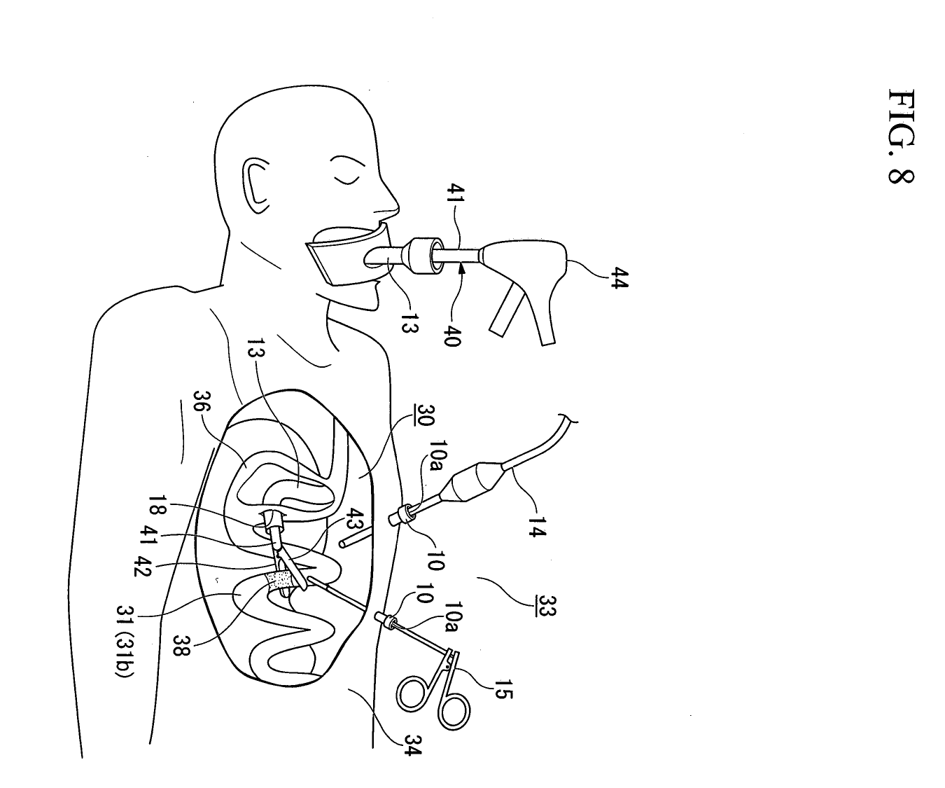

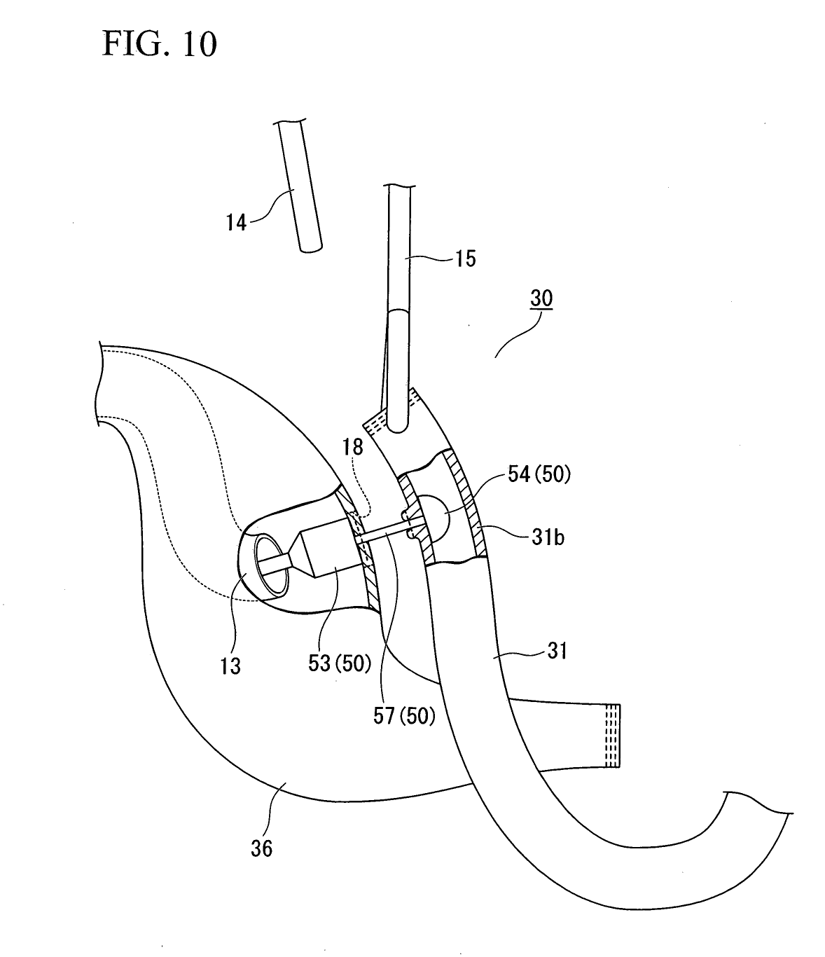

FIGS. 8 through 12 show a first variant example of this embodiment, and show an example of an anastomosis operation in which a small bowel 31 is anastomosed to a stomach 36 (i.e., a stomach—small bowel bypass) using an automatic suturing and cutting instrument 40 and an anastomosis instrument 50. This type of medical procedure may be performed in order to treat obesity.

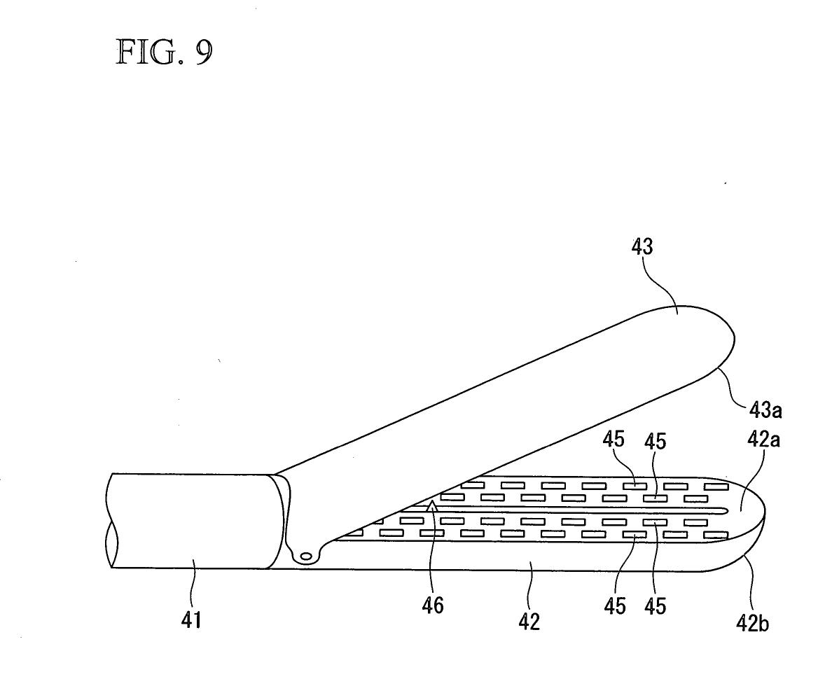

As shown in FIG. 9, the automatic suturing and cutting instrument 40 has a flexible sheath 41 that is able to be inserted into the overtube 13, a cartridge 42 and anvil 43 that are provided at a distal end portion of the sheath 41, and an operating section 44 that is provided at a proximal end portion of the sheath 41. The cartridge 42 is fixed to a distal end portion of the sheath 41 while the anvil 43 is attached by a shaft such that it can be opened and closed on the cartridge 42 by an operation of the operating section 44. Namely, the cartridge 42 and the anvil 43 are able to nip an object using their respective nipping surfaces 42a and 43a as the result of an operation of the operating section 44. A plurality of staples 45 are incorporated aligned in the axial direction in the nipping surface 42a of the cartridge 42, and it is possible to drive the staples 45 into a nipped object. Furthermore, a movable cutter 46 that is able to cut an object that is nipped by the cartridge 42 and the anvil 43 along the axial direction is provided between the plurality of staples 43. An observation apparatus may also be provided in a distal end portion 42b.

As shown in FIG. 8, firstly, the grasping forceps 15 are inserted through a trocar 10 and are made to grasp an area adjacent to a portion for excision 38. It is also possible for the portion for excision 38 itself to be grasped, or for both the portion for excision 38 and an area adjacent thereto to be grasped. Next, the automatic suturing and cutting instrument 40 is inserted orally using the overtube 13 into the stomach 36, and is made to protrude into the abdominal cavity 30 through the through hole 18 that was formed previously. Based on observations made using the laparoscope 14, an area adjacent to the portion for excision 38 where an excision is to be made using the automatic suturing and cutting instrument 40 is then grasped by the grasping forceps 15. In addition, the excision of the portion for excision 38 and the suturing of end portions of that portion 31b that is to be bypassed to the stomach 36 (referred to below as the “object portion”) are performed by the automatic suturing and cutting instrument 40. Namely, the portion for excision 38 of the small bowel 31 is nipped by the cartridge 42 and the anvil 43 of the automatic suturing and cutting instrument 40. Next, staples 45 are driven into the nipped portion for excision 38 of the small bowel 31 by an operation of the operating section 44, and the small bowel 31 is cut between the driven staples by an operation of the cutter 46. By then performing the stapling on both sides of the cutter 46, the portion for excision 38 is excised and end portions of the remaining object portion 31b are sutured. Next, the automatic suturing and cutting instrument 40 is withdrawn from the overtube 13.

Here, because the automatic suturing and cutting instrument 40 is introduced into the abdominal cavity 30 via a natural aperture 30, it is possible to keep the diameter of the insertion hole 10a that is formed in the abdominal wall 34 to the minimum hole diameter size of 5 mm or less that enables the grasping forceps 15 to be inserted. Moreover, the automatic suturing and cutting instrument 40 that requires a hole diameter of 10 mm or more can be introduced orally into the abdominal cavity 30.

Next, as shown in FIGS. 10 through 12, using the anastomosis instrument 50 the object portion 31b of the small bowel 31 is anastomosed to the stomach 36 so that a bypass is formed between the stomach 36 and the small bowel 31.

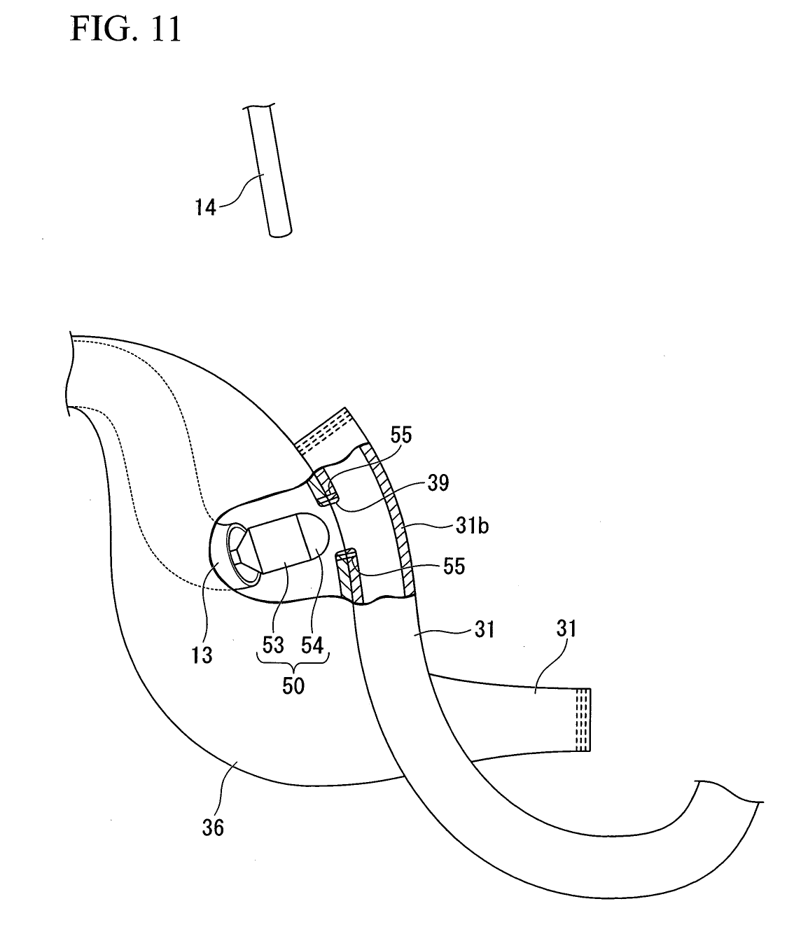

As shown in FIG. 12, the anastomosis instrument 50 has a flexible sheath 51, and a substantially columnar anastomosis portion 52 that is provided at a distal end portion of the sheath 51. An operating section is also provided at a proximal end portion (not shown) of the sheath 51. The anastomosis portion 52 has a substantially cylindrical staple driving portion 53 that is provided at a distal end portion of the sheath 51 and, based on the operation of the operating section, is able to drive in a plurality of staples 55 in a circular pattern from a nipping surface 53a that is formed at a distal end thereof, and an anvil portion 54 that serves as a shaping device. A cutter 56 that has a substantially cylindrical shape and is able to be moved reciprocatingly in an axial direction by the operation of the operating section is provided in an interior portion of the staple driving portion 53. The anvil portion 54 is joined to the staple driving portion 53 side by an anvil shaft 57 that penetrates the staple driving portion 53 and is inserted as far as the sheath 51. More specifically, the anvil shaft 57 is inserted through the sheath 51 and is connected to an operating wire that is connected to the operating section. The anvil shaft 57 is thus able to be moved reciprocatingly together with the operating wire by an operation of the operating section. As a result, an object can be nipped in a toroidal shape by the nipping surfaces 53a and 54a of the staple driving portion 53 and the anvil portion 54.

Next, a detailed description will be given of the anastomosis of the stomach 36 and the object portion 31b of the small bowel 31 using the above described anastomosis instrument 50. Firstly, the anastomosis instrument 50 is inserted inside the overtube 13 that has been orally inserted into a living body and whose distal end has been introduced into the abdominal cavity 30 via the through hole 18. The anvil portion 54 and the staple driving portion 53 of the anastomosis instrument 50 are then made to protrude from the distal end of the overtube 13. The anvil portion 54 is then introduced through the through hole 18 into the interior of the abdominal cavity 30, and the circumference of the through hole 18 that surrounds the anvil shaft 57 is sutured using a suturing instrument that has been inserted through an insertion hole 10a. Next, using an incision instrument (for example, shearing forceps) that has been inserted through an insertion hole 10a, an incision is made in the object portion 31b of the small bowel 31 that is to be anastomosed to the stomach 36, and the anvil 54 is placed inside the object portion 31b. The position where the incision is made may be the portion cut by the automatic suturing and cutting instrument 40 or may be adjacent thereto. Once the anvil portion 54 has been placed inside the object portion 31b, the area surrounding the anvil shaft 57 is sutured using the suturing instrument that has been inserted through an insertion hole 10a.

Once the anvil portion 54 has been placed inside the object portion 31b, the operating section is operated so that the anvil portion 54 is pulled towards the staple driving portion 53 side, and the tissue being anastomosed is sandwiched between the staple driving portion 53 and the anvil portion 54. A cutting operation to form a connecting hole 39 that connects the stomach 36 to the object portion 31b is then performed by further operating the operating section 54 so that the cutter 56 is moved forward and cuts the tissue between the stomach 36 and the small bowel 31. In addition, a suturing operation to suture the stomach 36 and the object portion 31b while simultaneously arresting any hemorrhaging is then performed by driving staples 55 from the staple driving portion 53 into the circumference of the connecting hole 39 that has been cut As a result, anastomosis of the stomach 36 and the object portion 31b of the small bowel 31 is achieved. The processing sequence to perform anastomosis using the grasping forceps 15 and the like can also be assisted by making observations using the laparoscope 14.

As has been described above, it is also possible when performing an anastomosis to keep the diameter of the insertion hole 10a that is formed in the abdominal wall 34 to the minimum hole diameter size of 5 mm or less that enables the grasping forceps 15 to be inserted. Moreover, the anastomosis instrument 50 that requires a hole diameter of 15 mm or more can be introduced orally into the abdominal cavity 30 so that the stomach 36 and the small bowel 31 can be anastomosed.

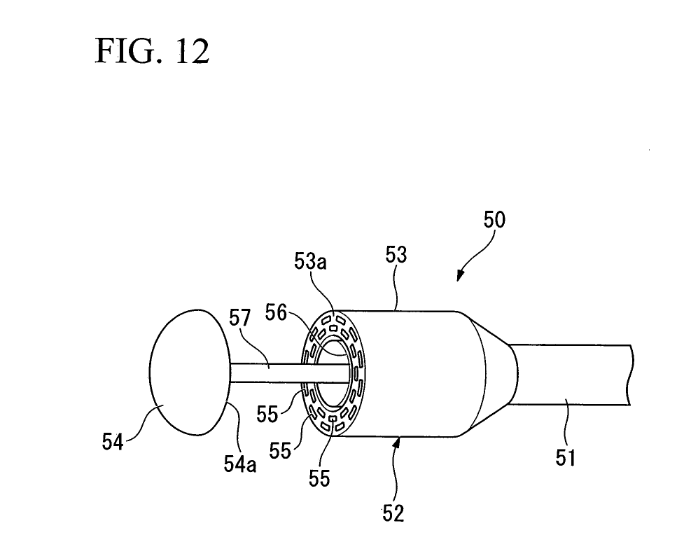

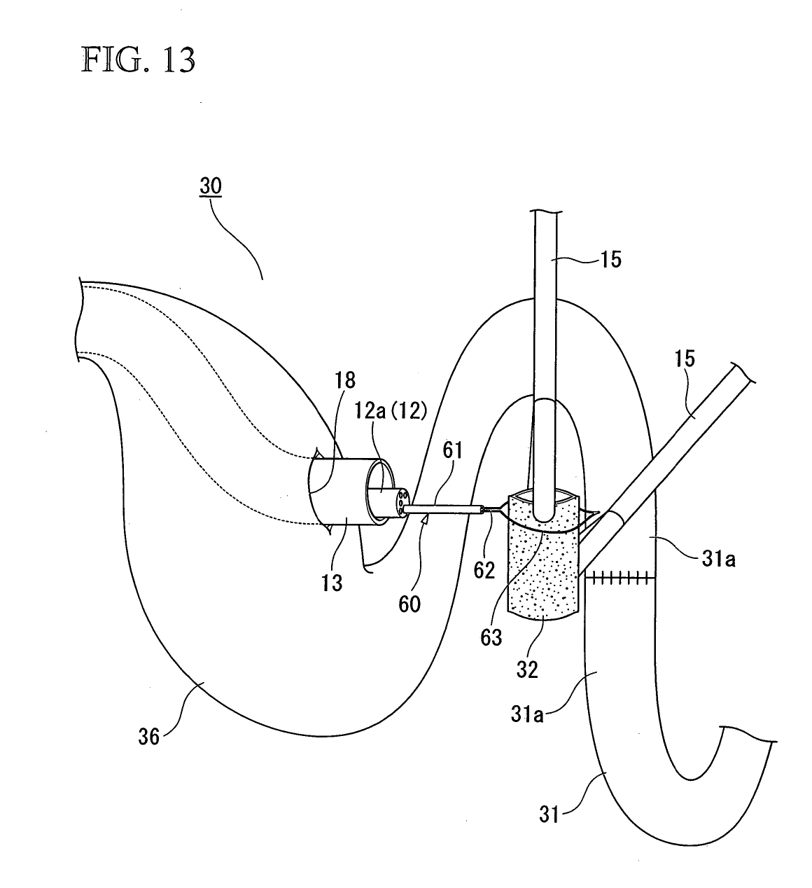

FIGS. 13 and 14 show a second variant example of this embodiment, and show a variant example in which, after the cut lesioned portion 32 has been cut up finely (i.e., after the target location (i.e., the retrieval object) has been divided into a plurality of pieces), the pieces are removed using a retrieval instrument that is introduced orally into the abdominal cavity 30. Depending on the type of medical procedure that is performed inside the abdominal cavity 30, there may be cases in which the size of the retrieval object (i.e., a lesioned portion or organ such as a gall bladder) that is to be removed to the body exterior is too large to be removed orally in its existing state. In a conventional laparoscopic operation, if the retrieval object that is to be removed to the body exterior is large in size, then because it is not possible for it to be removed to the body exterior via a trocar, an incision is made in the abdomen corresponding to the size of the retrieval object, and after the retrieval object has been removed, the incised portion is sutured. In the present variant example, when removing an object to the body exterior, the retrieval object is removed to the body exterior without making an incision in the abdomen but by cutting the object into pieces small enough to allow them to be removed perorally.

As shown in FIG. 13, in this variant example, two grasping forceps 15 are inserted through trocars and grasp the lesioned portion 32. In addition, a high frequency snare 60 is inserted perorally to serve as a cutting instrument.

The high frequency snare 60 has a sheath 61 that can be inserted inside the channel 12c of the endoscope 12, a conductive operating wire 62 that is inserted through the sheath 61, and a toroidal snare 63 that is provided at a distal end portion of the operating wire 62. The snare 63 is conductive and is also resilient. An operating section (not shown) is provided at a proximal end portion of the sheath 61, and the operating wire 62 can be moved reciprocatingly through the operation of the operating section. Because of this, by moving the operating wire 62 backward using the operating section the snare 63 can be accommodated in the sheath 61, and by moving the operating wire 62 forward the snare 63 can be made to protrude from the sheath 61 and the diameter thereof can also be enlarged due to its own resiliency. An electrode is also provided in the operating section, and by connecting this to a power supply a high frequency current can be supplied to the snare 63 via the operating wire 62.

Firstly, the endoscope 12 is inserted through the overtube 13 and the distal end 12a is made to protrude through the through hole 18 into the abdominal cavity 30. The high frequency snare 60 is then inserted through the channel 12c of the endoscope 12 and is made to protrude from the distal end 12a. Next, by operating the operating section of the high frequency snare 60, the snare 63 is made to protrude from the sheath 61 and the extirpated lesioned portion 32 is contained inside the ring formed by the snare 63. If, as shown in FIG. 14, the operating wire 62 is then moved backward by the operating section, the snare 63 is pulled inside the sheath 61 and its diameter is contracted so that it tightens around the lesioned portion 32. If, at the same time as this, high frequency current is supplied by the operating section to the snare 63, the lesioned portion 32 is burnt through so as to form a section 32a that has been cut to a small size. If the cut section 32a is cut to a size that allows it to be placed inside a retrieval instrument such as the retrieval net 19 or a retrieval bag and then removed perorally to the body exterior, then it is possible to remove perorally to the body exterior a retrieval object such as a lesioned portion or organ that is too large to pass through the overtube 13. FIG. 14 shows a state in which the section 32a that has been formed by cutting the lesioned portion 32 is grasped by the grasping forceps 15. The endoscope 12 is subsequently removed, and in its place a retrieval instrument such as the retrieval net 19 shown in FIG. 4 or a retrieval bag is introduced via the overtube 13 into the abdominal cavity 30, thereby enabling the task of retrieval to be performed. By then repeating the task of cutting the lesioned portion 32 into small pieces and then removing these using a retrieval instrument through the overtube 13, it is possible to remove the entire lesioned portion 32. When, in view of post-surgical scarring, trocars having insertion holes 10a that have small hole diameters are used and the sections 32a are removed to the body exterior via these insertion holes 10a, then the sections 32a need to be cut even smaller, however, by retrieving them through the patient's mouth, they can be removed to the body exterior while being left at a comparatively larger size. Accordingly, a pathological diagnosis after the operation can be performed more efficiently.

As described above, in this variant example as well, it is possible to keep the diameter of the insertion hole 10a that is formed in the abdominal wall 34 to the minimum hole diameter size of 5 mm or less that enables the grasping forceps 15 to be inserted. Furthermore, it is possible to cut the extirpated lesioned portion 32 into small sections 32a using the high frequency snare 60, and it is possible to remove a retrieval object perorally to a body exterior even when the retrieval object is large.

Note that in the present variant example, the high frequency snare 60 is used as an example of a cutting instrument for cutting the lesioned portion 32 into sections, however, the present invention is not limited to this and it is also possible to select a variety of instruments in accordance with the size and hardness of the object. For example, instead of the high frequency snare 60, it is also possible to divide the object into a plurality of sections using a cutting instrument that is provided with a plurality of loop-shaped portions that serve as cutting portions that cut tissue, and that, when the object for cutting is placed inside the plurality of loop-shaped portions, is able to cut the object into a plurality of small sections by contracting the diameter of the respective loop-shaped portions. It is also possible to finely mince the object using an apparatus that is able to chop the object into a minced form. Moreover, the cutting of a retrieval object such as a lesioned portion or organ may also be performed using a cutting instrument that has been inserted through a trocar. In this case, by removing the cut object through a natural aperture, as shown in FIG. 4, substantially the same effects as those of the above described variant example can be obtained. It is also possible instead of the high frequency snare 60 to use a treatment instrument that is provided with a plurality of wires formed in a basket shape, and that is able to crush an object contained therein by expanding and contracting these wires. In these cases as well, because the retrieval of the retrieval object is achieved using a retrieval instrument that is perorally introduced into the abdominal cavity 30, it is not necessary for the diameter of the insertion holes 10a that are formed in the abdominal wall 34 to be enlarged.

An embodiment of the present invention is described above in detail with reference made to the drawings, however, the specific structure thereof is not limited to this embodiment and other design modifications and the like can be made thereto without departing from the spirit or scope of the present invention.

Note also that in the present embodiment and the variant examples thereof, a description is given of an example of a medical procedure in which a lesioned portion 32 of a small bowel 31 is cut and sutured, and the lesioned portion 32 is also removed to the body exterior 33, however, the present invention is not limited to this. It is at least possible for the same effects to be anticipated in a medical procedure that is performed inside the abdominal cavity 30. For example, the same effects can be expected when a gall bladder or appendix or the like is removed to the body exterior, or when a lesioned portion or the like that is formed on another organ within the abdominal cavity 30 such as a kidney or pancreas is removed.

Furthermore, in the second treatment step, a second apparatus was inserted through the mouth 35 of a patient and was introduced into the interior of the abdominal cavity 30 through the through hole 18 that was formed in the stomach 36, however, the present invention is not limited to this. It is also possible for the nose or anus to be used as the aperture through which the second apparatus is inserted in the second treatment step, and by at least inserting the second apparatus through a natural aperture, it is possible to reduce the diameter of the insertion holes 10a through which the first apparatus is inserted percutaneously in the first treatment step. Moreover, it is also possible to form a through hole in an organ other than the stomach 36 so that an apparatus can be introduced into the abdominal cavity 30.

What is claimed is:

1. A medical procedure performed inside an abdominal cavity comprising:
performing a first treatment at a target position inside the abdominal cavity by a first apparatus that has been introduced percutaneously into the abdominal cavity; and
performing a second treatment, using a second apparatus that has been introduced into the abdominal cavity via a natural aperture of a living body, in cooperation with the first apparatus inside the abdominal cavity, or alternatively, performing a second treatment that is necessitated as a result of the first treatment being performed after the first treatment has been performed.

2. The medical procedure performed inside an abdominal cavity according to claim 1, further comprising: forming a through hole, which is used to introduce the second apparatus into the abdominal cavity, in a hollow organ that is connected with the natural aperture; removing the second apparatus from inside the abdominal cavity after the second treatment has been performed; and closing, after the removal, the connection between an inner side of the hollow organ in which the through hole is formed and the abdominal cavity.

3. The medical procedure performed inside an abdominal cavity according to claim 2, wherein the closing of the connection between an inner side of the hollow organ and the abdominal cavity includes suturing the through hole using a suturing instrument that is introduced percutaneously into the abdominal cavity.

4. The medical procedure performed inside an abdominal cavity according to claim 1, comprising percutaneously inserting a laparoscope into the abdominal cavity, wherein the first treatment and the second treatment are performed while being observed using the laparoscope.

5. The medical procedure performed inside an abdominal cavity according to claim 2, wherein the introducing of the first apparatus into the abdominal cavity includes introducing the first apparatus into the abdominal cavity through an insertion hole that is formed in the abdominal wall and has a hole diameter of 5 mm or less, and the introducing the second apparatus into the abdominal cavity includes introducing apparatuses, which require a hole having a larger hole diameter than 5 mm to be formed when they are introduced into the abdominal cavity, into the abdominal cavity via the through hole.

6. The medical procedure performed inside an abdominal cavity according to claim 1, wherein in the first treatment, the first apparatus is inserted into the abdominal cavity through an insertion hole that is formed in the abdominal wall having a hole diameter of a size that can be closed not by suture closure but by natural closure, and if it is necessary to insert an apparatus into the abdominal cavity through a hole diameter that is too large to be closed by natural closure, then in the second treatment the apparatus is inserted into the abdominal cavity through a natural aperture.

7. The medical procedure performed inside an abdominal cavity according to claim 1, wherein the target position is an organ in the abdominal cavity.

8. The medical procedure performed inside an abdominal cavity according to claim 1, wherein the performing of the first treatment includes cutting the target position using the first apparatus that has been introduced into the abdominal cavity percutaneously.

9. The medical procedure performed inside an abdominal cavity according to claim 8, further comprising forming a through hole, which is used to introduce the second apparatus into the abdominal cavity, in a hollow organ that is connected to the natural aperture, wherein the second treatment includes: using at least a retrieval instrument that is introduced into the abdominal cavity through the through hole; retrieving the target position that was cut in the first treatment as a retrieval object; and removing the target position to the body exterior through the natural aperture.

10. The medical procedure performed inside an abdominal cavity according to claim 8, further comprising dividing the target position, which was cut in the first treatment, into a plurality of sections, wherein the second treatment includes removing the plurality of sections into which the target position was divided to the body exterior using at least the retrieval instrument.

11. The medical procedure performed inside an abdominal cavity according to claim 1, wherein the second treatment includes cutting of the target position and suturing of the circumference of the cut portion using an automatic suturing and cutting instru ment that has been introduced into the abdominal cavity via a natural aperture of the living body, and the first treatment includes grasping at least one of the target position and the vicinity of the target position using a grasping instrument that has been introduced percutaneously so that the suturing of the cut at the target position and the circumference of the cut portion by the automatic suturing and cutting instrument is assisted.

12. The medical procedure performed inside an abdominal cavity according to claim 1, wherein the second treatment includes anastomosing an area between different positions of a single organ or an area between different organs using an anastomosis instrument that has been introduced into the abdominal cavity via a natural aperture in the living body, and the first treatment includes grasping at least one of the target position and the vicinity of the target position using a grasping instrument that has been introduced percutaneously so that the anastomosis by the anastomosis instrument is assisted.
